(19) United States
(12) Patent Application Publication
(10) Pub. No.: US 2022/0079827 A1
(43) Pub. Date: Mar. 17, 2022

(54) FLUID COLLECTION DEVICE, RELATED APPARATUS AND METHOD OF USING THE SAME

(71) Applicant: Vimadri, LLC, Salt Lake City, UT (US)

(72) Inventors: Joseph LOMBARDO, Salt Lake City, UT (US); Alyssa Ann Varghese, Galveston, TX (US); Erica Arnold, Salt Lake City, UT (US)

(73) Assignee: Vimadri, LLC, Salt Lake City, UT (US)

(21) Appl. No.: 17/475,471

(22) Filed: Sep. 15, 2021

Related U.S. Application Data
(60) Provisional application No. 63/078,506, filed on Sep. 15, 2020.

(51) Int. Cl. A61G 13/10; A61B 17/42; A61B 46/20

(52) U.S. Cl. CPC A61G 13/102; A61B 2090/063; A61B 46/20; A61B 17/42

(57) ABSTRACT

A device for collecting fluid discharged from a vagina of a woman with the woman seated or lying on a support surface, the device including a receptacle portion for receiving the fluid and a planar portion for inserting under the woman. The receptacle portion has an angled bottom surface and a drain opening for draining fluid from the receptacle portion. Also, an apparatus containing the device and a vessel for collecting and measuring fluid drained from the vessel and a method for collecting and measuring fluid loss by a woman following birth of her child by vaginal delivery.

### CROSS REFERENCE TO RELATED APPLICATION

This application claims priority to U.S. Application Ser. No. 63/078506 filed on Sep. 15, 2020 entitled “Fluid Collection Receptacle”, which application is incorporated herein by reference.

### FIELD OF THE INVENTION

The invention relates to a receptacle for the collection of fluid and specifically for a receptacle comprising a planar portion that is configured to insert under the body of a woman who has given birth to a child by vaginal delivery and a receptacle portion to receive fluid that is discharged from the birth canal of the woman. The invention also relates to an apparatus that comprises the receptacle and means for draining, collecting and measuring the amount of fluid received in the receptacle. The invention further relates to a method of measuring the amount of fluid discharged from the birth canal of the woman following the vaginal delivery.

### BACKGROUND OF THE INVENTION

Postpartum hemorrhage (also called PPH) is a condition marked by a woman having heavy bleeding after giving birth. It's a serious but rare condition that usually occurs within 1 day of giving birth. About 1 to 5 in 100 women who have a baby (1 to 5 percent) have PPH.

Postpartum hemorrhage is defined as blood loss that is greater or equal to 500 mL within the first 24 hours after vaginal delivery.

Photospectometry is the most accurate technique for measuring postpartum blood loss. However, photospectometry is expensive and often impractical. The more commonly used method of measuring blood loss is to use a sterile under-buttock drape that contains a receptacle with a graded measurement scale comprised of a series of marks usually representing milliliters. This is described, for example, in international publication WO/2014/083573 for an invention entitled “Surgical Drape with Calibrated Pouch for Collecting and Measuring Fluid”, the contents of which are incorporated herein by reference. The volume of fluid collected in the receptacle, as represented by a fluid mark reached in the receptacle of the drape, is intended to give an indication of the amount of blood loss.

Unfortunately, the measurement provided by conventional surgical drapes is often inaccurate due to the presence in the drape receptacle of fluids other than blood. Although the drape is intended to be placed under a woman immediately following delivery of her child, as a practical matter the drape is usually put in place before the delivery since the positioning of a flexible drape with attached receptacle under a woman is a time-consuming and labor-intensive process insofar as it involves moving the woman from her position on a delivery table or bed while the drape is put in place. The pre-delivery positioning of the drape leads to other materials, such as amniotic fluid, urine, irrigation, meconium, vernix and, occasionally, feces, being collected within the drape receptacle. These other materials may lead to clogging the receptacle, not allowing the collection of subsequent fluids. Fluids may also not be collected within the drape receptacle and may instead be excreted outside of the receptacle. Additionally, the angle at which the receptacle lies, and thus the mouth size of the receptacle, is completely dependent on the placement of the drapes and so may not be conducive to fluid collection. These factors can result in inaccurate measurement of blood loss which in turn results in poor early detection of maternal hemorrhage.

What is needed is a receptacle which can be placed easily and swiftly under a woman directly following delivery without repositioning the woman so as to collect only the fluid discharged from the woman's birth canal and which can also provide for the collection and accurate measurement of the volume of blood discharged from the birth canal.

### SUMMARY OF THE INVENTION

In a first embodiment of the invention, there is provided a device for collecting fluid discharged from a vagina of a woman with the woman seated or lying on a support surface, the device comprising:

a receptacle portion for receiving the fluid and a planar portion for inserting under the woman, the receptacle portion comprising (a) a bottom surface, (b) at least one wall surrounding the bottom surface, and (b) an opening for draining fluid received in the receptacle portion; the bottom surface of the receptacle portion being contoured to guide fluid received in the receptacle portion toward the opening;

the planar portion comprising a front edge that is connected to a top edge of a rear segment of the at least one wall of the receptacle portion, the planar portion being sufficiently thin and rigid to be slid under the woman with the woman seated or lying on the support surface with her vagina disposed over an edge of the support surface, the planar portion and the receptacle portion being configured such that, with the planar portion slid under the woman, the planar portion sits atop the support surface and is retained in place at least in part by the weight of the woman and the receptacle portion is disposable adjacent the edge of the support surface with the bottom surface of the receptacle portion under the support surface and under the vagina of the woman to receive fluid discharged from the vagina.

In a preferred aspect of this embodiment, the planar portion has a bottom surface comprising a plurality of protrusions for helping to retain the device in place when it is slid under the woman. Preferably, each of the protrusions protrudes from the planar surface at an acute angle in a direction toward the receptacle portion. In a most preferred embodiment, the plurality of protrusions extend across an entire width of the planar portion and are arranged to form parallel ridges on the bottom surface of the planar portion.

In another preferred aspect of this embodiment, the drain opening is disposed in a first side segment of the at least one wall of the receptacle portion adjacent the bottom surface and the bottom surface is angled from a second side segment of the at least one wall to the first side segment, wherein the second side segment is on an opposite side of the receptacle portion from the first side segment such that the bottom surface guides fluid received in the receptacle portion to the port. The angle of the bottom surface is preferably between 5 and 25 degrees relative to horizontal.

In yet another preferred aspect of this embodiment, the front edge of the planar portion forms an angle with the top edge of the rear segment of the at least one wall of the receptacle portion of between 90 and 120 degrees.

In a further preferred aspect of this embodiment, the device comprises a spout in fluid connection to the drain opening for conducting fluid out of the receptacle portion.

In a still further preferred aspect of this embodiment, the planar portion has a thickness of less than 0.125 inches and a Young modulus of at least 0.5 GPa, and the planar portion has a length as measured along the front edge of between 12 and 24 inches and a width as measured from the front edge to a back edge of the planar portion of between 4 and 8 inches.

In yet another preferred aspect of this embodiment, the rear segment of the receptacle portion is substantially straight and the receptacle portion has a front segment opposed to the rear segment that bows away from the rear segment, and the receptacle portion has a length as measured from the first side segment to the second side segment of between 12 and 24 inches and a width as measured from the rear segment to a part of the front segment that is furthest away from the rear segment of between 4 and 8 inches. Preferably, the part of the front segment that is furthest away from the rear segment is in the middle of the front segment.

In a most preferred aspect of this embodiment, the device is made of a disposable plastic material and the disposable plastic material is selected from the group consisting of polypropylene, polyethylene, and polyethylene terephthalate. Preferably, the device has no sharp edges and is coated with an anti-bacterial agent.

In a second embodiment of the invention, the invention comprises an apparatus comprising the aforementioned device, a vessel for collecting fluid drained from the receptacle portion and means connected to the spout for conducting fluid drained from the receptacle portion to the vessel. Preferably, the means for conducting fluid comprises tubing and suction means for sucking fluid through the tubing into the vessel. Most preferably, the vessel comprises a graduated scale to facilitate measurement of the fluid collected in the vessel.

In a third embodiment of the invention, there is provided a method for collecting and measuring fluid loss by a woman following birth of her child by vaginal delivery, wherein the woman is sitting or lying on a support surface, the method comprising the steps of:

(a) providing the aforementioned;

(b) sliding the device of the apparatus under the body of the woman immediately following the delivery of the child such that the planar portion of the device sits flat atop the support surface and is retained in place by the weight of the woman and the receptacle portion of the device is disposed adjacent an edge of the support surface with the bottom surface of the receptacle portion under the support surface and under the vagina of the woman to receive fluid discharging from the vagina;

(c) causing the fluid received in the receptacle portion to drain from the device through the tubing and into the vessel of the apparatus; and

(d) measuring the amount of fluid in the vessel.

In a preferred aspect of this embodiment, the vessel comprises a graduated scale that provides an indication of how much fluid is in the vessel and the measuring comprises comparing a level of the fluid in the vessel to the graduated scale.

In another preferred aspect of this embodiment, the woman is lying on her back on the support surface in the dorsal lithotomy position.

In yet a further aspect of this embodiment, the woman is disposed on the support surface in a seated or semi-seated position.

### BRIEF DESCRIPTION OF THE DRAWINGS

FIG. 1 is a perspective view of the device of the invention in a first embodiment.

FIG. 2 is a top plan view of the device shown in FIG. 1;

FIG. 3 is a sectional view of the device taken along line D-D of FIG. 2;

FIG. 4 is a front view of the device shown in FIG. 1;

FIG. 5 is a sectional view of the device taken along line E-E of FIG. 4:

FIG. 6 is a right side view of the device shown in FIG. 1:

FIG. 7 is a detailed view of a drain opening and port of the device of FIG. 1;

FIG. 8 is a detailed view of a side wall of the device of FIG. 1;

FIG. 9 is a perspective view of the device of the invention in a second embodiment;

FIG. 10 is a top plan view of the device shown in FIG. 9;

FIG. 11 is a front view of the device shown in FIG. 9;

FIG. 12 is a sectional view of the device taken along line I-I of FIG. 11;

FIG. 13 is a side view of the device shown in FIG. 9;

FIG. 14 is a sectional view of the device taken along line K-K of FIG. 10;

FIG. 15 is a bottom view of the device shown in FIG. 9:

FIG. 16 is a detailed side view of the planar portion of the device shown in FIG. 9 with protrusions;

FIG. 17 is a detailed side view of one of the protrusions; and

FIG. 18 is a perspective view of a preferred apparatus of the invention in position to receive, collect and measure fluid discharged from the birth canal of a woman post-delivery.

### DETAILED DESCRIPTION

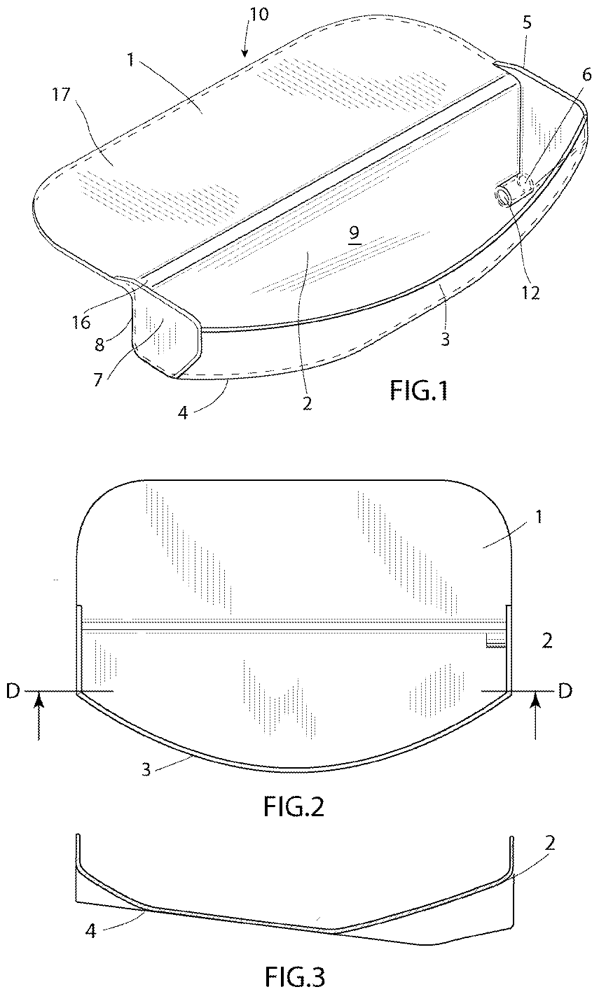

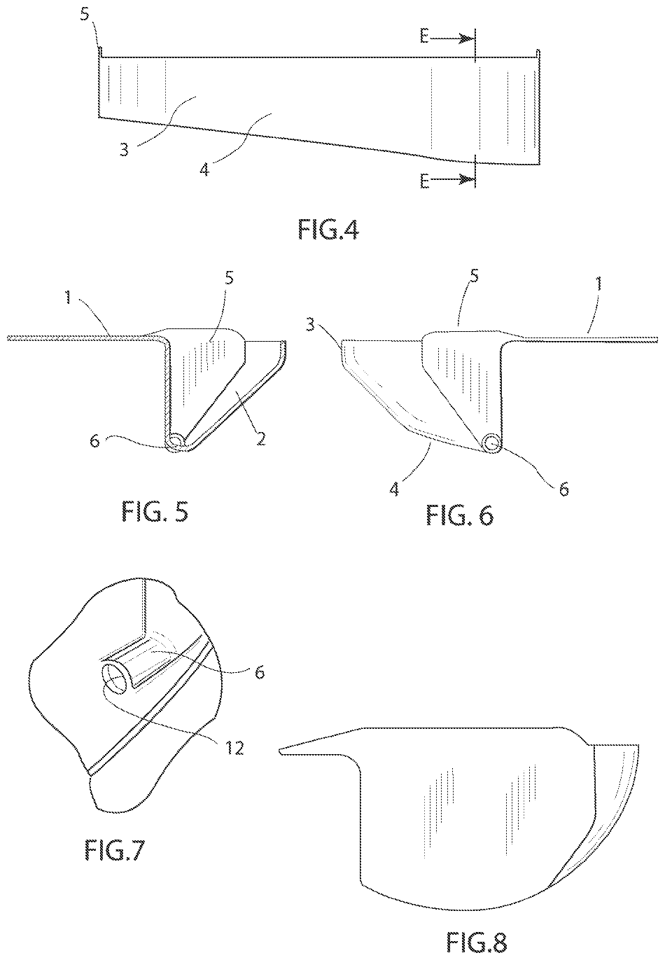

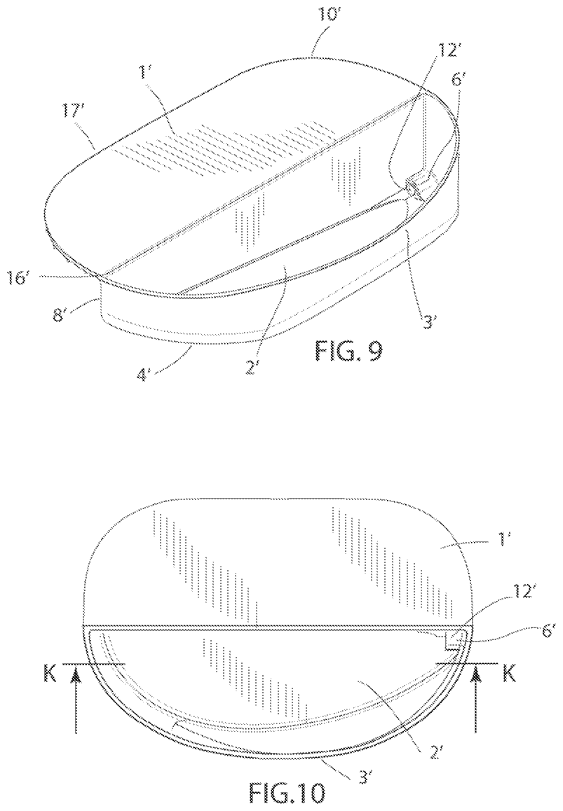

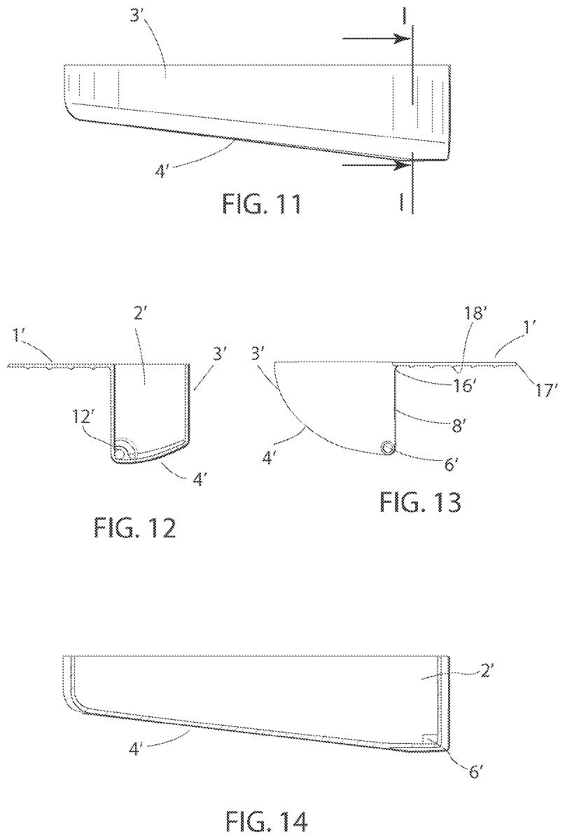

Referring to the drawings, FIGS. 1-8 show one embodiment of a device 10 for positioning under a woman post-delivery for receiving blood discharged from the birth canal of the woman. The device 10 comprises a planar portion 1 for inserting under the woman and a receptacle portion 2 for receiving the discharged blood. In the embodiment shown in FIG. 1, the receptacle portion 2 comprises four (4) walls 3, 5, 7 and 8 and a bottom surface 4 defining a cavity 9 for receiving the fluid. Although the embodiment shown in FIG. 1 has four (4) distinct walls, namely left sidewall 7, right sidewall 5, front wall 3 and back wall 8, it is contemplated that the receptacle portion 2 of device 10 can also be formed with a single wall of, for example, oblong shape without corners defining separate walls, as further discussed below with respect to the embodiment shown in FIGS. 9-17. In that event, the single wall will nevertheless have opposed sidewall segments and opposed front and back wall segments that are functionally equivalent to the distinct walls shown in FIG. 1.

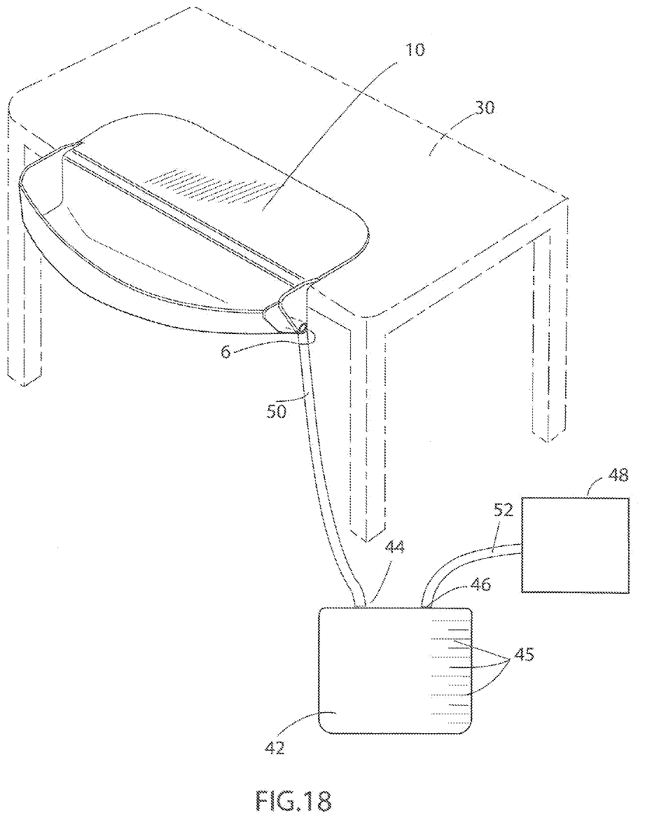

As shown in FIG. 1, a drain opening 12 is formed in the right sidewall 5 to allow blood to flow out of the cavity 9 through suction tubing 50 to a suction canister 42, as shown in FIG. 18 and explained more fully below. The drain opening 12 is surrounded by a port or spout 6, which may be connected by suction tubing 50 to the suction canister 42. The bottom surface 4 or a portion thereof is angled or tapered from left side wall 7 to the drain opening 12 in right side wall 5. The angled surface is used to direct fluid toward the drain opening 12. In effect, the angled surface or surface portion acts as a funnel to direct blood toward the drain opening 12 so that the fluid is not allowed to pool or settle in cavity 9. Rather, the blood will flow or be directed toward the drain opening 12 for removal through the drain opening.

FIG. 6 is a right side view of the device 10. The device 10 is shown having a drain spout or output port 6 extending out from the right sidewall 6. The spout 6 is adapted to be connected to a tube, a suction tube, or other tubing or conduit, all of which can direct blood out of the receptacle portion of device 10 and into a collection vessel, such as suction canister 42 (see FIG. 18), where the amount of blood received in the receptacle portion can be measured. The spout 6 is in fluid communication with the drain opening 12. The angled bottom surface 4 of the receptacle portion 2 works with the suction to direct or guide blood to the drain opening 12 and out through spout 6. The slope of the bottom surface 4 of the receptacle portion is preferably between 5 and 25 degrees relative to horizontal, is more preferably between 5 and 10 degrees relative to horizontal and is most preferably about 7 degrees relative to horizontal.

Referring to FIG. 7, the port 6 is shown as disposed in the interior of the receptacle portion 2. Alternatively, the port 6 can be an external port that protrudes out of the receptacle portion. An interior port is advantageous for a few reasons: i) an external port sticks out and could break off, ii) the device is more compact and streamlined with an internal port, and iii) with the internal port, the length of the device is uniform, whereas an external port increases the device length in that region.

Reference is now made to FIGS. 9-17 which depict the inventive device in a preferred embodiment having features which correspond generally to the features in the embodiment depicted in FIGS. 1-8, as shown by the use of corresponding reference numerals in the drawings to depict corresponding features. The embodiment of FIGS. 9-17 differs from the previously described embodiment in two (2) respects. First, rather than comprising four (4) walls, the device shown in FIGS. 9-17 comprises only a back wall 8′ and a single front/side wall 3′ that is arcuate in shape and extends from one end of the back wall 8′ to the other end of the back wall 8′. The arcuate front/side wall is preferably a curve of irregular shape or spline, rather than a curve of fixed radius, to render the device less bulky.

This geometry of the device is preferred insofar as it provides for a sleeker device while still optimizing the amount of fluid that can be captured in the receptacle. The provision of a sleek design is advantageous insofar as a large chamber could get in the way of space needed for a physician to place his or her hands to access the vaginal canal appropriately. Even after the neonate is delivered, the physician will need access to the canal for the delivery of the placenta and possible need for vaginal repair. The back wall 8′ in this embodiment, as with the back wall 8 of the previously described embodiment, is preferably flat so that it can rest securely against a flat side edge of a delivery table or bed when the device 10, 10′ is disposed at the edge of a delivery table with the planar portion 10, 10′ under a subject.

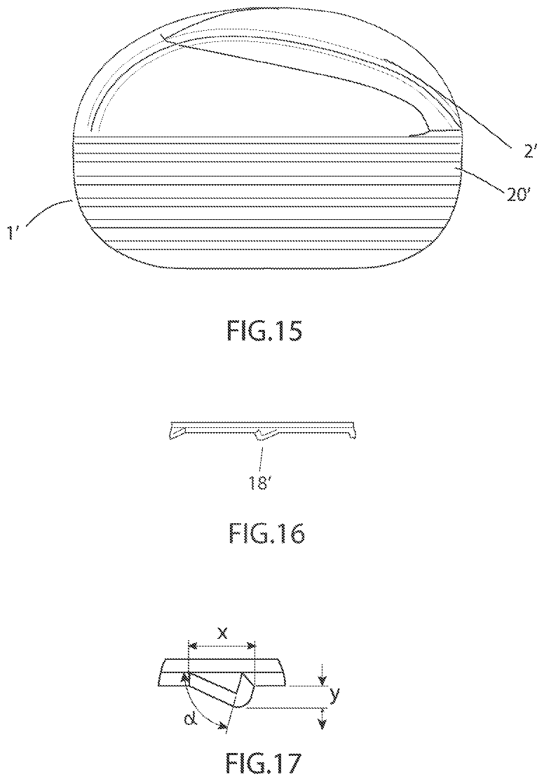

The embodiment of FIGS. 9-17 also differs from the device of FIGS. 1-8 in the provision of protrusions 18′ on the bottom surface 4′ of the device. The provision of protrusions 18′ helps to prevent the device from sliding out after placement and, in this respect, acts as a supplement to maintaining the position of the device by the body weight of a patient alone. As shown in FIGS. 16 and 17, the protrusions preferably extend from the planar portion 1′ toward the receptacle portion 2′ and protrude from the surface of the planar portion 2′ at an angle alpha of between about 60 and 85 degrees and more preferably at angle alpha of between about 70 and 75 degrees. This disposition of the protrusions maximizes the resistance required for the planar portion 2′ to move toward the receptacle portion 1′, which is desirable to retain the device in place, and minimizes the resistance when the planar portion 2′ is slid in the opposite direction, which is desirable when sliding the device under the body of a woman. The length x of each of the protrusions is preferably between about 0.15 and 0.5 inches, and the height y of each of the protrusions is preferably about 0.1 inches (see FIG. 17). As shown in FIG. 15, in a preferred embodiment, each of the protrusions extends across the entire width of the planar portion 1′ and the plurality of protrusions are preferably disposed to form parallel ridges 20′ that can provide maximum friction to help to prevent movement of the device 10, 10′ once the device is slid into place under the body of a woman.

As will be understood by one of skill in the art with reference to FIG. 18, when the device of the invention 10, 10′ is disposed to collect blood discharged from the vagina of a woman at the edge of a delivery table or like support 30, the blood collected in device 10, 10′will move across the angled bottom surface 4, 4′ of cavity 9, 9′ to drain opening 12, 12′ and port 6, 6′ where it is suctioned through suction tubing 50 to suction canister 42. The force of suction assists the angle of the bottom surface 4, 4′ in guiding the blood to the port 6, 6′ and thereafter to the suction canister 42. The suction canister 42 has at least two outlets 44 and 46 for tubing. One of the tubes 52 is attached to a suction source 48 and the other tube 50 is attached to the port 5, 6′ of device 10, 10′. The suction source 48 (which may be, for example, a wall outlet or a portable compressor) generates a negative pressure in the canister 42 that helps to dram the blood from the device 10, 10′. The suction canister 42 has graduated markings 45 which can be used to ascertain the amount of blood collected in the canister. The apparatus of the invention can operate within a normal delivery room or operating room conditions in unison with standard hospital suction collection systems.

As shown in FIGS. 1 and 9, the planar portion 1, 1′ of devices 10 and 10′ has a front edge that is connected to a top edge of back wall 8, 8′ of the receptacle portion to form joint edge 16, 16′. The walls of the receptacle portion 2, 2′ form an angle with respect to the surface of planar portion 1, 1′ such that, with the planar portion disposed under the body of a woman seated or lying with her vagina disposed near an edge of the delivery surface, the receptacle portion can be positioned below the delivery surface (see FIG. 18) to receive blood discharged from the woman's vagina. To achieve this preferred disposition of the respective planar and receptacle portions, the angle between the respective portions should be between about 90 and 120 degrees, preferably between about 90 and 100 degrees and most preferably about 90 degrees.

The planar portion 1, 1′ and receptacle portion 2, 2′ are preferably integrally formed and the device is preferably of unitary construction. The device is preferably made of a disposable or reusable plastic material although, in addition to polymers, they may be made of other materials, such as metal or glass. Preferably, the device 10, 10′ is made of a polymer such as polyethylene or polypropylene. If the material of the device is reusable, it will be sterilized according to standard hospital requirements between patients. The sterilized device will be delivered in sterile packaging to the site of use. The material should not be bioactive with human skin and may be coated with an antibacterial agent.

Regardless of the material with which the device is made, the planar portion 1, 1′ should be sufficiently thin and rigid to enable it to be slid under the body of a woman lying or seated on a delivery surface. For example, with a woman lying on her back in the dorsal lithotomy position, her legs flexed 90 degrees at the hips, her knees bent at 70 to 90 degrees, and her feet positioned in padded foot rests to support her legs, the planar portion should be slidable under the back of the woman's buttocks without the need to reposition her. As another example, with the woman seated or semi-recumbent on a delivery bed, table or chair with her feet raised, the planar portion should be slidable under the bottom of the women's buttocks without the need to reposition her. To provide this functionality, the planar portion will preferably have a uniform thickness of between 0.08 and 0.125 inches and will preferably have a Young modulus of between 0.5 and 2 GPa.

In order to slide the planar portion 1, 1′ under a woman's buttocks while the woman is seated or lying on a delivery surface with her vagina positioned over the edge of the delivery surface, the receptacle portion 2, 2′ of device 10, 10′ can be grasped by the delivery provider at or near the front of the receptacle portion. The delivery provider can then push the device towards the woman's buttocks so that the top edge 17, 17′ of the planar portion 1, 1′ reaches the buttocks first. The pressure exerted by the delivery provider on the receptacle portion 2, 2′ toward the woman, and if necessary or desirable the woman's own strength to pull up and lift her buttocks, allows the planar portion of the device to slide under the woman's body without the need for her to move from her position on the delivery surface.

The device is configured and dimensioned such that, once placed under the woman's body, the planar portion receives the back or the bottom of the woman's buttocks with the receptacle portion aligned under the woman's vagina at the edge of the delivery surface to receive blood discharged from the birth canal. The weight of the woman's body, paired with the protrusions 18′ or anti-slip ridges 20, can maintain the device 10 in this fixed alignment. Preferably, the planar portion of the device has a length, extending from side wall segment 6, 6′ to side wall segment 7, 7′, of between about 12 to 24 inches and more preferably between about 16 and 20 inches. The planar portion 1, 1′ preferably has a width, extending from joint edge 16, 16′ to top edge 17, 17′ of the planar portion of between about 4 and 8 inches and more preferably between about 5 and 7 inches. The length is preferably greater than the width.

The receptable portion 2, 2′ preferably has a length, extending from sidewall section to sidewall section, that is the same as the width of the planar portion. The receptacle portion is shaped to optimize blood collection with the front wall 3, 3′ having a bowed or rounded shape for optimal blood collection with the width of the receptacle portion preferably being greatest in the middle of the rounded wall. Preferably, the width of the receptacle portion 2, 2′, as measured from the joint edge 16, 16′ to the middle of the rounded wall 3, 3′ is between about 4 and 8 inches and is more preferably between about 5 and 7 inches, The length is preferably greater than the width.

The device of the invention can be positioned on the edge of any suitable delivery support, including a hospital bed, a delivery table or a delivery chair. The delivery support preferably has at least a surface portion that is flat and a side edge that is flat such that the planar portion 1, 1′ of the device can lie flat atop the flat surface portion of the delivery surface and the flat back wall of the receptacle portion 2, 2′ can lie flat against the flat side edge of the delivery support. When a hospital bed is used as the delivery surface, the foot board of the hospital bed may be removed for the delivery.

Those skilled in the art will readily observe that numerous modifications and alterations of the device and method described herein and shown in the drawings may be made while retaining the teachings of the invention. Accordingly, the present disclosure should be construed as limited only by the metes and bounds of the appended claims and then equivalents.

What is claimed is:

1. A device for collecting fluid discharged from a vagina of a woman with the woman seated or lying on a support surface, the device comprising:
a receptacle portion for receiving the fluid and a planar portion for inserting under the woman, the receptacle portion comprising (a) a bottom surface, (b) at least one wall surrounding the bottom surface, and (b) a port for draining fluid received in the receptacle portion; the bottom surface of the receptacle portion being contoured to guide fluid received in the receptacle portion toward the port;
the planar portion comprising a front edge that is connected to a top edge of a rear segment of the at least one wall of the receptacle portion, the planar portion being sufficiently thin and rigid to be slid under the woman with the woman seated or lying on the support surface with her vagina disposed atop an edge of the support surface, the planar portion and the receptacle portion being configured such that, with the planar portion slid under the woman, the planar portion sits atop the support surface and is retained in place at least in part by the weight of the woman and the receptacle portion is disposable adjacent the edge of the support surface with the bottom surface of the receptacle portion under the support surface and under the vagina of the woman to receive fluid discharged from the vagina.

2. The device according to claim 1, wherein the drain opening is disposed in a first side segment of the at least one wall of the receptacle portion adjacent the bottom surface and the bottom surface is angled from a second side segment of the at least one wall to the first side segment wherein the second side segment is on an opposite side of the receptacle portion from the first side segment such that the bottom surface guides fluid received in the receptacle portion to the port.

3. The device according to claim 2, herein the bottom surface is angled at between about 5 and 35 degrees relative to horizontal.

4. The device according to claim 3, comprising a spout in fluid connection to the drain opening for conducting fluid out of the receptacle portion.

5. The device according to claim 4, wherein the planar portion has a thickness of less than 0.125 inches and a Young's modulus of at least 0.5 GPa.

6. The device according to claim 5, wherein the planar portion has a length as measured along the front edge of between 12 and 24 inches and a width as measured from the front edge to a back edge of the planar portion of between 4 and 8 inches.

7. The device according to claim 6, wherein the rear segment of the receptacle portion is substantially straight and the receptacle portion has a front segment opposed to the rear segment that bows away from the rear segment.

8. The device according to claim 7, wherein the receptacle portion has a length as measured from the first side segment to the second side segment of between 12 and 24 inches and a width as measured from the rear segment to a part of the front segment that is furthest away from the rear segment of between 4 and 8 inches.

9. The device according to claim 8, wherein the part of the front segment that is furthest away from the rear segment is in the middle of the front segment.

10. The device according to claim 9, wherein the device is made of a disposable plastic material selected from the group consisting of polypropylene, polyethylene, and polyethylene terephthalate.

11. The device according to claim 1, wherein the planar portion has a bottom surface comprising a plurality of protrusions that are angled from the bottom surface of the planar portion toward the receptacle portion for helping to retain the device in place when it is slid under the woman.

12. The device according to claim 11, wherein the plurality of protrusions are arranged to form parallel ridges on the bottom surface of the planar portion.

13. The device according to claim 2, wherein the device is coated with an anti-bacterial agent.

14. The device according to claim 2, wherein the front edge of the planar portion forms an angle with the top edge of the rear segment of the at least one wall of the receptacle portion of between 90 and 120 degrees.

15. An apparatus comprising the device according to claim 3, a vessel for collecting fluid drained from the receptacle portion and means connected to the spout for conducting fluid drained from the receptacle portion to the vessel.

16. The apparatus according to claim 15, wherein the means for conducting fluid comprising tubing and suction means for sucking fluid through the tubing.

17. The apparatus according to claim 15, wherein the vessel has a graduated scale to facilitate measurement of the fluid drained from the receptacle portion.

18. A method for collecting and measuring fluid loss by a woman following birth of her child by vaginal delivery, wherein the woman is sitting or lying on a support surface, the method comprising the steps of:
(a) providing the apparatus of claim 15;
(b) sliding the device under the body of the woman immediately following the delivery of the child such that the planar portion of the device sits flat atop the support surface and is retained in place by the weight of the woman and anti-slip ridges, and the receptacle portion of the device is disposed adjacent air edge of the support surface with the bottom surface of the receptacle portion under the support surface and under the vagina of the woman to receive fluid discharging from the vagina;
(c) causing the fluid received in the receptacle portion to drain from the device into the vessel; and
(d) measuring the amount of fluid in the vessel.

19. The method according to claim 18, wherein the vessel comprises a graduated scale that indicates how much fluid is in the vessel and the measuring comprises comparing a level of the fluid in the vessel to the graduated scale.

20. The method according to claim 19, wherein the woman is disposed on the support surface in a dorsal lithotomy or semi-seated position.
